(19) United States
(12) Patent Application Publication
(10) Pub. No.: US 2013/0281916 A1
(43) Pub. Date: Oct. 24, 2013

(54) SONOSENSITIVE NANOPARTICLES

(71) Applicants: Sarah Jayne Wagstaffe, Oxford (GB); Heiko Alexander Schiffter-Weinle, Oxford (GB); Michael Bernard Molinari, Oxford (GB); Manish Arora, Oxford (GB); Coussios Constantin-Cassios, Oxford (GB)

(72) Inventors: Sarah Jayne Wagstaffe, Oxford (GB); Heiko Alexander Schiffter-Weinle, Oxford (GB); Michael Bernard Molinari, Oxford (GB); Manish Arora, Oxford (GB); Coussios Constantin-Cassios, Oxford (GB)

(73) Assignee: lsis Innovation Limited, Oxford ,Oxfordshire (GB)

(21) Appl. No.: 13/885,565

(22) PCT Filed: Nov. 17, 2011
(86) PCT No.: PCT/GB2011/052244

§ 371 Date: Jul. 3, 2013

(30) Foreign Application Priority Data
Nov. 17, 2010 (GB) 1019434.8

(51) Int. Cl. A61K 41/00; A61K 49/22

(52) U.S. Cl. CPC A61K 41/0033; A61K 41/0028; A61K 49/222
USPC 604/22; 424/489; 428/402; 428/407

(57) ABSTRACT

A method of delivering a therapeutic substance to tissue comprises delivering the therapeutic substance and nanoparticles to the tissue, the nanoparticles having a diameter in the range from 10 to 1000 nm and surface features having a depth in the range from 5 to 50 nm, and insonating the tissue with pressure waves. Corresponding particles, and associated methods of controlling and imaging the treatment and delivery are also disclosed.

### FIELD OF THE INVENTION

The present invention relates to nanoparticles and their use in initiating acoustic cavitation when exposed to pressure waves such as ultrasound.

### BACKGROUND TO THE INVENTION

The significance of cavitation for therapeutic ultrasound processes has long been known, as has the difficulty of instigating cavitation in vivo. The most common approach used to date to lower the cavitation threshold is the use of injectable microbubbles stabilized by a lipid or protein shell (also known as ultrasound contrast agents). Even though these agents will lower the threshold, their size makes them unsuitable for accumulation in the microcirculation and particularly in tumours. Because they encapsulate gas, these microbubbles will also change their size and behaviour over a period of hours in the body.

It is also known to form bubbles by means of acoustic droplet vaporisation (see http://www.ultrasound.med.umich.edu/Projects/ADV.html). In this process superheated droplets of, for example, perfluorocarbon, generally in the nanometer range, are expanded upon acoustic excitation to provide a vapour-filled bubble. The disadvantages are that their stability in vivo has yet to be demonstrated; and the fact that the bubble produced is essentially a vapour bubble, which means that it is difficult to cause it to collapse inertially. It may therefore not be possible to cause the therapeutic bioeffects associated with inertial cavitation using this method.

### SUMMARY OF THE INVENTION

Methods of manufacturing rough-surfaced nanoparticles that can, amongst other things, facilitate the initiation of acoustic cavitation when exposed to diagnostic or therapeutic ultrasound.

The primary application is intended to be the combination of therapeutic ultrasound with those nanoparticles for targeted drug delivery. Acoustic cavitation, the non-linear oscillation of gas- and vapour-filled cavities under the effect of a sound field, has been shown to play a key role in several therapeutic ultrasound applications, including targeted drug release and delivery, and the extravasation of therapeutic agents from the blood stream into surrounding tissues.

However, there are few nuclei naturally available within the body to seed acoustic cavitation, and as a result extremely large pressure amplitudes are required in order to initiate this process. Some embodiments of the invention aim at producing biocompatible nanoparticles, of the right size to naturally accumulate in tumours (10-1000 nm), which have the right surface characteristics (e.g. hydrophobicity and surface roughness) to facilitate inception of cavitation under ultrasound excitation. These particles may be used to simply lower the cavitation threshold in therapeutic ultrasound applications where cavitation has been found to play a significant role, such as non-invasive ablation by high intensity focussed ultrasound, ultrasound-enhanced thrombolysis, sonoporation, sonophoresis, opening of the blood-brain barrier, lithotripsy. However, those sonosensitive nanoparticles may either be attached to or combined with a drug, vaccine or other therapeutic agent to also enable cavitation-mediated targeted drug release and delivery.

The desired particles may be obtained by any suitable method. Preferably they are manufactured by one of the following techniques (but not limited to): layer-by-layer assembly, spray-freeze-drying, emulsification techniques (such as single and double emulsion techniques), emulsion diffusion, polymer coacervation, nanoprecipitation and spray-drying. The final particles may have a particle diameter between 10 nm and 1000 nm (measured e.g. by dynamic light scattering, scanning electron microscopy, transmission electron microscopy, or other suitable particle size determination methods). Particle populations can show a monodisperse or polydisperse size distribution. Particles can have a hollow or solid core. Particles have a rough surface morphology (determined by scanning electron microscopy) with a surface area larger than that of an ideal sphere (determined by gas adsorption, BET). Particles can consist of a range of materials including, but not limited to (i) natural and synthetic biocompatible and/or biodegradable polymers such as poly(lactic acid), poly(lactic-co-glycolic acid), poly(caprolactone), poly(ethylene glycol), (ii) inorganic material such as gold, silicon and titanium dioxide, etc.

The use of nanometer-sized carriers for targeted drug release by ultrasound is also known. These carriers generally currently take the form of liposomes or micelles. Liposomal carriers can be thermosensitive, i.e. have a shell that becomes leaky at a given temperature following heating. Such carriers necessitate a significant energy input using therapeutic ultrasound in order to release their payload. Other liposomal or micellar carriers can be made to release their payload by rupture of the liposome or micelle through mechanical forces, possibly caused by cavitation (see Evjen T J, Nilssen E A, Røgnvaldsson S, Brandl M, Fossheim S L. Distearoylethanolamine-based liposomes for ultrasound-mediated drug delivery. Eur J Pharm Biopharm. 2010; 75(3):327-33), but because the carrier itself has no effect on the local cavitation threshold, the pressure amplitudes required to achieve this are known to be very high (above 15 MPa in the MHz frequency range).

The present invention can, in some embodiments, enable localized alteration of the cavitation threshold in microvascularized tissues using stable, solid nanoparticles. Because cavitation is a pressure driven rather than an energy driven phenomenon, short pulses of ultrasound that exceed the cavitation threshold may be sufficient to release encapsulated drugs. This may therefore provide a low energy release mechanism by ultrasound.

The invention is expected to have widespread applicability throughout the field of therapeutic ultrasound, as described above. Applications beyond healthcare but in combination with acoustic waves could include sonochemistry, chemical engineering, or any other application where facilitating inception of acoustic cavitation is important.

The science underlying the invention is the identification of methods that can suitably alter the hydrophobicity and surface roughness of nanoparticles so as to entrap minute amounts of gas in order to facilitate cavitation inception when exposed to negative pressures. Furthermore, a recent combined experimental and numerical study of acoustic cavitation in tissue (S. Labouret and C-C. Coussios, J. Acoust. Soc. Am, 2011, in press) has demonstrated that, subject to certain assumptions about the surface tension and viscoelastic properties seen by cavitation nuclei in tissue, nuclei of particular sizes are more likely to cavitate inertially at particular excitation frequencies. In particular, the study has shown that in order for cavitation activity to occur for single-cycle acoustic excitation at pressure amplitudes below 3 MPa peak negative, nuclei smaller or equal to 8 nm are required at 1.6 MHz, smaller or equal to 11 nm are required at 1 MHz, and smaller or equal to 28 nm are required at 0.5 MHz.

The present invention makes use of this in that, in embodiments of the invention, if nuclei of particular sizes are formed on the surface of rough nanoparticles, they will respond preferentially at particular excitation frequencies.

The present invention therefore further pertains to methods of tuning the hydrophobicity and surface roughness of nanoparticles so as to entrap minute amounts of gas of specific size ranges in order to facilitate cavitation inception when exposed to negative pressures at particular ultrasound frequencies.

The present invention therefore provides a nanoparticle for inducing cavitation in a medium under insonation, the nanoparticle having a diameter no more than 1000 nm, and optionally in the range from 10 to 1000 nm, and surface features having a depth no more than 50 nm, and optionally in the range from 5 to 50 nm.

The present invention further provides a nanoparticle for the treatment of cancer in a body, the nanoparticle having a diameter no more than 1000 nm, and optionally in the range from 10 to 1000 nm and surface features having a depth no more than 50 nm, and optionally in the range from 5 to 50 nm, whereby the nanoparticle is arranged to enhance cavitation in the body when the body is insonated with pressure waves.

The present invention further provides a system for treating cancerous tissue, the system comprising a source of pressure waves and nanoparticles for delivery to the tissue, the nanoparticles having a diameter no more than 1000 nm, and optionally in the range from 10 to 1000 nm and surface features having a depth no more than 50 nm, and optionally in the range from 5 to 50 nm, whereby the nanoparticles are arranged to enhance cavitation in the tissue when the tissue is insonated with pressure waves from the source.

The present invention further provides a method of controlling cavitation in tissue, the method comprising delivering nanoparticles to the tissue, the nanoparticles having a diameter no more than 1000 nm, and optionally in the range from 10 to 1000 nm and surface features having a depth no more than 50 nm, and optionally in the range from 5 to 50 nm, and insonating the tissue with pressure waves.

The present invention further provides a method of imaging an object, the method comprising delivering nanoparticles to the object, the nanoparticles having a diameter no more than 1000 nm, and optionally in the range from 10 to 1000 nm and surface features having a depth no more than 50 nm, and optionally in the range from 5 to 50 nm, and insonating the object with pressure waves such that the nanoparticles induce cavitation in the object, detecting pressure waves generated by the cavitation by means of a detector, and processing signals from the detector to generate an image of the object and/or of the cavitating region.

The present invention further provides a method of monitoring the delivery of a therapeutic substance to tissue, the method comprising delivering the therapeutic substance and nanoparticles to the tissue, the nanoparticles having a diameter no more than 1000 nm, and optionally in the range from 10 to 1000 nm and surface features having a depth no more than 50 nm, and optionally in the range from 5 to 50 nm, and insonating the tissue with pressure waves such that the nanoparticles induce cavitation in the tissue, detecting pressure waves generated by the cavitation by means of a detector, and processing signals from the detector to monitor the delivery.

The present invention further provides a method of delivering a therapeutic substance to tissue, the method comprising delivering the therapeutic substance and nanoparticles to the tissue, the nanoparticles having a diameter no more than 1000 nm, and optionally in the range from 10 to 1000 nm and surface features having a depth no more than 50 nm, and optionally in the range from 5 to 50 nm and insonating the tissue with pressure waves.

The present invention further provides a method of both mapping the location of the nanoparticles and of delivering a therapeutic substance to tissue. This may be achieved using nanoparticles having two or more characteristic lengthscales of cavitation nuclei: a first nucleation lengthscale that responds at a particular excitation frequency to induce cavitation activity for the purposes of imaging the location of the particles, and a second lengthscale that responds at a different excitation frequency in order to cause inertial cavitation that enhances delivery of the therapeutic substance, for example by rupturing the nanoparticle, resulting in the delivery of the enclosed therapeutic substance. In some embodiments a mixture of two groups of nanoparticles each having surface characteristics at one of the lengthscales can be used, and in some embodiments nanoparticles each of which has surface features at both of the lengthscales can be used.

The preferred particle diameteter is determined by physiological factors. Particles up to 1000 nm will be readily taken up by general microvasculature (such as that involved in brain drug delivery applications). However particles smaller than about 600 nm are readily retained by leaky tumour vasculature (under the Enhanced Permeability and Retention effect or EPR). Particles of diameter 100-300 nm are preferentially taken up by tumour vasculature. Therefore in all of the above cases, the nanoparticles may have a diameter not more than 800 nm, for example the diameter may be in the range 50-800 nm. In some cases the diameter may be not more than 600 nm, for example it may be in the range 50-600 nm. In some cases the diameter may be not more than 300 nm, for example it may be in the range 50-300 nm. In some cases it may be preferable for the diameter to be at least 100 nm, for example it may be in the range 100-300 nm. The diameter may be measured as the mean diameter, for example the volume moment mean diameter of the particle D(4, 3).

The surface features may be formed by particles, which may be spheres or part-spheres. The particles may have a diameter of not more than 50 nm, for example in the range 5-50 nm. The surface features may be formed by depressions with depth of not more than 50 nm, for example in the range 5-50 nm, and or width not more than 50 nm, for example in the range of 5-50 nm, in order to match the preferred nucleus size for acoustic cavitation at particular ultrasound excitation frequencies.

In all of the above methods and systems, the ultrasound may have a frequency of at least 100 kHz. Indeed the ultrasound in each case may have a frequency within the range 100 kHz to 10 MHz. In each case the ultrasound frequency may be at least 500 kHz. In each case, the frequency may be in the range 500 kHz to 5 MHz.

The nanoparticles may be hydrophobic, their hydrophobicity being primarily determined by the choice of material forming the outer layer of the nanoparticle. Such materials should therefore exhibit a high contact angle, ideally greater than 60 degrees, as measured by one of the following methods: the static sessile drop method; the dynamic sessile drop method, the dynamic Wilhelmy method, the single-fiber Wilhelmy method or the powder contact angle method. Materials with a contact angle equal to or in excess of the contact angle for silicon dioxide are therefore preferred.

The nanoparticles may carry a drug. For example the nanoparticles may be hollow forming nanocapsules containing the drug, or the drug may be incorporated into the structure of the nanoparticles.

The nanoparticles may be freeze-dried or spray-freeze-dried or spray-dried. This may be done after a drug is encapsulated in the particles. This can provide the surface features of the required scale. In other cases other surface modification methods can be used to ensure that the surface features of the required scale are present.

The nanoparticles may be each formed by providing a core and forming a shell on the core. The core may be, for example, of polystyrene. The shell may be formed at least partly from silicon dioxide or titanium dioxide. The core may be removed to leave a hollow shell.

The shell may be formed at least partly from particles having a diameter in the range 5 to 50 nm so as to provide the surface features.

Preferred embodiments of the present invention will now be described by way of example only with reference to the accompanying drawings.

### BRIEF DESCRIPTION OF THE DRAWINGS

FIG. 1 is a diagram showing the steps in a method of making nanoparticles according to an embodiment of the invention;

FIG. 2 is a set of images of core-shell nanoparticles formed according to an embodiment of the invention;

FIG. 3 is a further set of images of core-shell nanoparticles formed according to an embodiment of the invention;

FIG. 4 is a further set of images of shell nanoparticles formed according to an embodiment of the invention;

FIG. 5 is a graph showing the probability of cavitation as a function of peak pressure for various substances;

FIG. 6 is a graph showing cavitation noise emissions as a function of peak pressure for various substances;

FIG. 7 is a graph showing probability of cavitation as a function of peak pressure for various substances;

FIG. 8 is a graph showing cavitation noise emissions as a function of peak pressure for various substances;

FIG. 9 is a set of images of particles formed by single emulsion;

FIG. 10 is a set of images of particles formed by single emulsion with camphor;

FIG. 11 is a set of images of particles formed by double emulsion;

FIG. 12 is a diagrammatic representation of a system operating according to an embodiment of the invention;

FIG. 13 is a chart showing cavitation threshold for water and blood containing nanoparticles according to an embodiment of the invention, and blood without nanoparticles;

FIGS. 14a, 14b and 14c are graphs showing probability of cavitation as a function of peak pressure at different ultrasound frequencies, for respective coating particle sizes; and

FIGS. 15a, 15b and 15c are graphs showing probability of cavitation as a function of peak pressure at different ultrasound frequencies, for respective nanoparticle sizes.

### DESCRIPTION OF THE PREFERRED EMBODIMENTS

Nanoparticles suitable for use in the present invention can be produced in a number of different ways, some examples of which will now be described.

### Layer-by-Layer Assembly

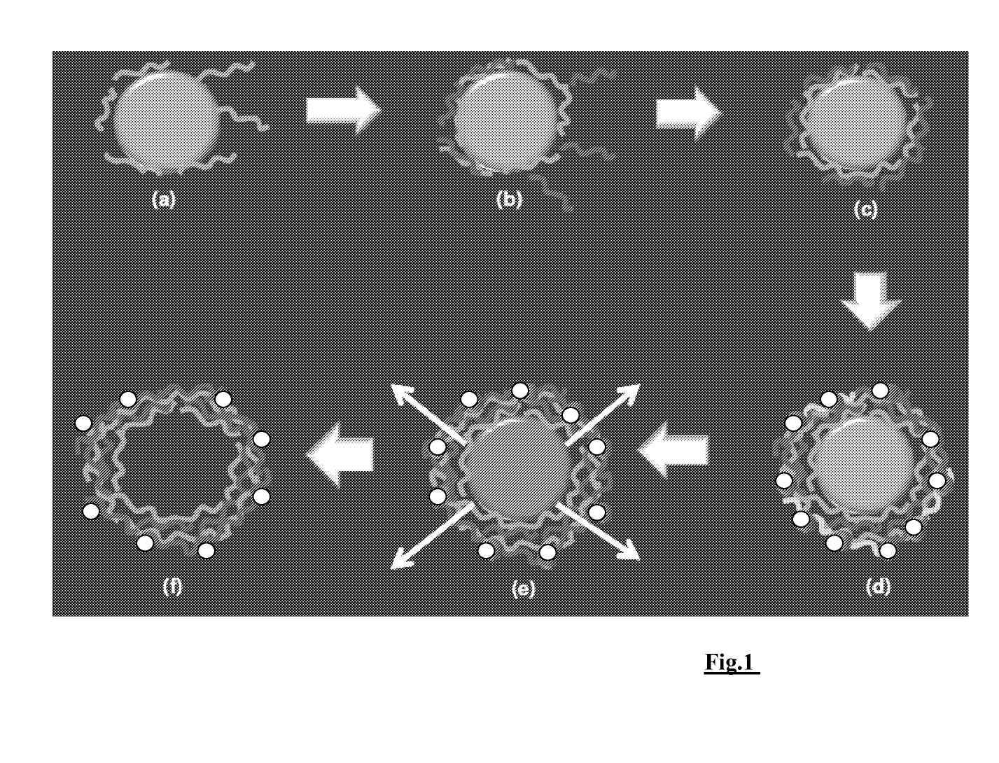

Referring to FIG. 1, nanoparticles according to one embodiment of the invention were prepared in a layer-by-layer assembly method. A suitable nanoparticle (e.g. polystyrene, gold etc.) in the size range of 20 nm to 600 nm was employed to act as a template for layer-by-layer polyelectrolyte deposition. The anionic template particles were suspended in solution with sonication prior to incubation with a cationic polymer polydiallyl dimethyl ammonium chloride (PDADMAC) in 0.5M NaCl causing PDADMAC deposition to the template surface as shown in FIG. 1(a). The particles were centrifuged at 13,500×g to form a nanoparticle pellet and resuspended in deionized water with sonication. The particles were washed a further two times in these conditions to remove excess PDADMAC. The particles were finally resuspended into a solution of an anionic polymer polystyrenesulfonate (PSS) in 0.5M NaCl and incubated to ensure PSS deposition to the cationic-coated surface as shown in FIG. 1(b). The solution was then centrifuged at 13,500×g once again to form a nanoparticle pellet as shown in FIG. 1(c). The particles were washed on a further two occasions to remove excess PSS. The particles were finally resuspended into a solution of the cationic polymer PDADMAC in 0.5M NaCl, and once again washed three times with deionized water to ensure excess electrolyte is removed from the particles. Final resuspension was into a solution of colloidal silica (LUDOX®), 40% solution in 0.1M NaCl. After incubation to ensure SiO₂ deposition to the cationic coated surface, as shown in FIG. 1(d) the particles were once again cleaned by repeated centrifugation-resuspension steps to remove excess silicon dioxide from the particles. The particles were subsequently cleaned and freeze-dried in order to remove any water and ensure air entrapment onto the particle surface.

The particles produced as described above, comprising a core and a shell, and having a surface roughness as a result of the SiO₂ particles deposited on the surface, can be used as they are. However inorganic (SiO₂/TiO₂) shell nanoparticles can be produced by calcination or chemical decomposition of the core shell rough surfaced nanoparticles as described above to remove the core.

In one embodiment, the rough surfaced particles produced above were made hollow by calcination or chemical decomposition of the core particle prior to the freeze drying step described. Particles were used either after furnace drying (calcination e.g. polystyrene core nanoparticle) or resuspended in deionized water or polymeric/surfactant stabilizer solutions prior to freeze drying (chemical decomposition of the core particle).

In one example of the method described above, in the preparation of 300 nm particles, 150 μl of 0.3 μm polystyrene latex was incubated with rotation with 30 ml PDADMAC (1 mg/ml, 0.5M NaCl) for 30 minutes to ensure polymer deposition to the polystyrene core surface. Samples were centrifuged at 13,500×g for 20 minutes then redispersed in an ultrasonic bath in deionized water before further centrifugation at 13,500×g for a further 20 minutes. This centrifugation-resuspension washing cycle was repeated a total of three times and final resuspension was into a 30 ml PSS solution (1 mg/ml in 0.5M NaCl). The samples were incubated for 20 minutes with rotation before washing by three repeated centrifugation-resuspension cleaning cycles described above. Resuspension after washing was into a second 30 ml PDADAMAC solution (1 mg/ml, 0.5M NaCl) for a further 20 minutes and particles were cleaned a further three times by centrifugation-resuspension cycles. Final resuspension was into a 30 ml LUDOX® solution (40% v/v solution in 0.1M NaCl) and incubation for 20 minutes preceded three final centrifugation-resuspension washing steps. The particles were then resuspended into 10 mls deionized water with sonication prior to freeze-drying in order to remove any water and ensure air entrapment onto the particle surface.

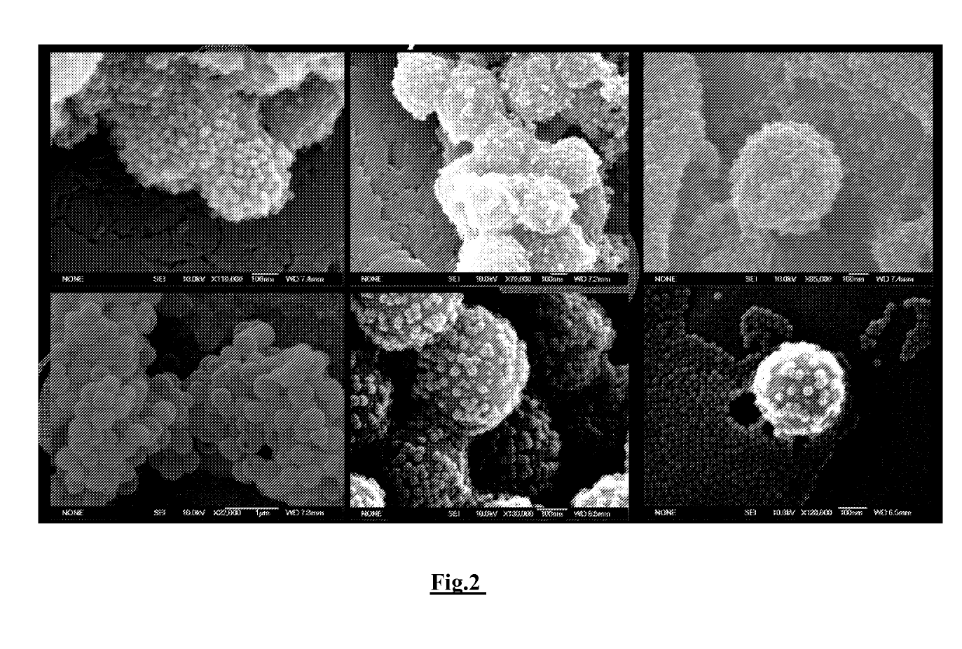

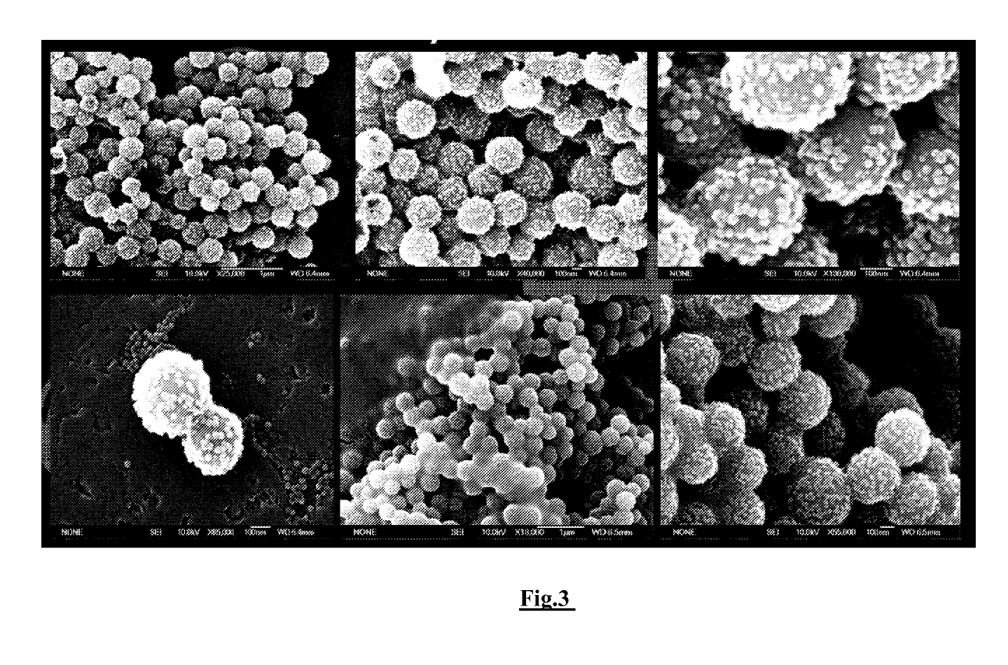

The resulting particles are shown in FIGS. 2 and 3, from which it can be seen that the particles had a diameter of about 300 nm as expected and the particles of SiO₂ are present on the surface of the nanoparticles and have a diameter of about 15-20 nm. This results in the surface of the nanoparticles having surface features having a depth in the range from 5 to 20 nm. For example where the SiO₂ forms part-spherical features on the relatively smooth surface of the underlying nano-sphere, the height of at least some of the features above the smooth surface is in the range 2 to 20 nm. Where the SiO₂ particles are closer together and form substantially the whole surface of the nano-sphere, each of the SiO₂ particles still forms a surface feature and has a depth within the 5-20 nm range. It will be appreciated that the surface of the SiO₂ particles will themselves have a surface roughness having surface features on a smaller scale.

Also the nanoparticles may, for example due to irregularities in the shape of the core or unevenness in the layer-on-layer process, have irregularities on a larger scale. However these do not significantly alter the effect of the surface roughness within the 5-20 nm range on the cavitation. Clearly of SiO₂ particles of different sizes were used, the nanoparticles can be given a surface roughness of a slightly different scale, for example up to 50 nm.

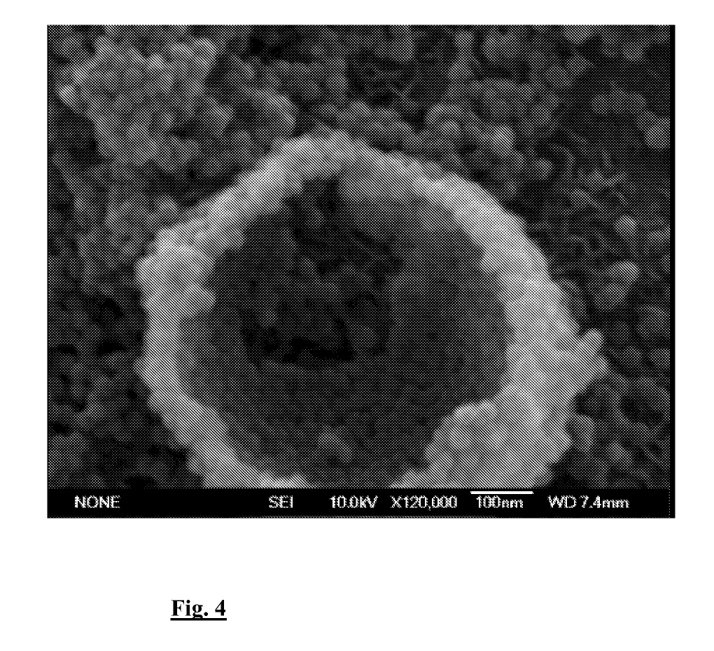

In an example of preparation of hollow inorganic (SiO₂/TiO₂) shell nanoparticles by calcination of polystyrene template from core-shell rough surfaced nanoparticles as described above, 5 mls of cleaned resuspended rough surfaced core-shell nanoparticles prepared above were heated in a controlled manner in an atmospheric environment to 600° C. and held at the final temperature for 4 hours to ensure complete calcination of the polystyrene core. The resulting particles are shown in FIG. 4, from which it can be seen that the nanoparticles are hollow with a shell formed mainly of SiO₂ particles which are in close contact, with the shell being two or three particles thick. Therefore in this case the surface roughness again has surface features each formed form part of one of the SiO₂ particles and having a depth of up to about the diameter of the SiO₂ particles, i.e. about 20 nm.

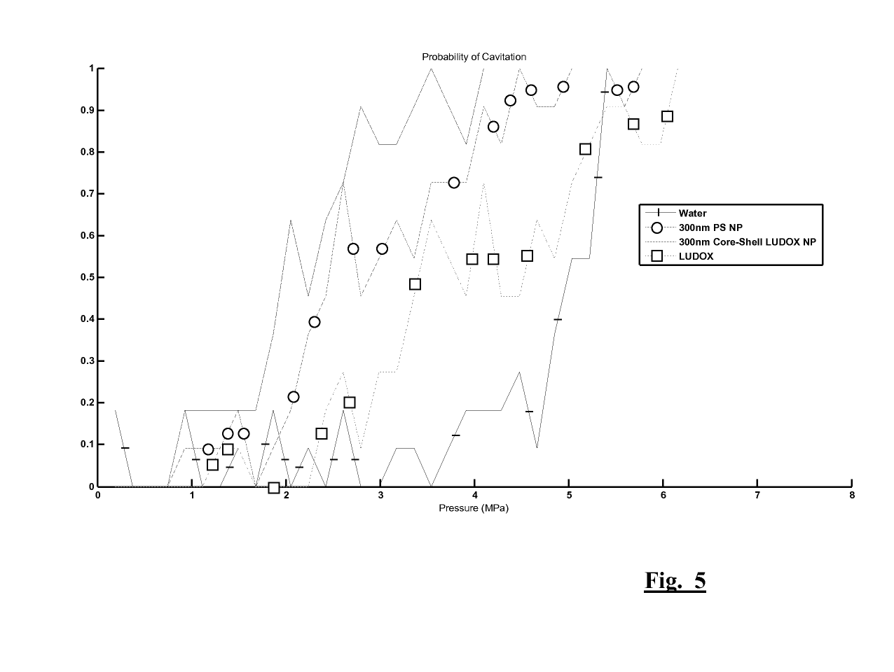

Referring to FIG. 5, various substances were insonated with 1 MHz therapeutic ultrasound and the probability of cavitation measured using an ultrasound detector array arranged to detect ultrasound produced by cavitation. The substances tested were plain water, water containing 300 nm particles formed by the layer-by-layer method without Ludox™, water containing 300 nm particles formed by the layer-by-layer method with Ludox™ to increase surface roughness, and plain Ludox. As can be seen, the presence of the nanoparticles enhanced cavitation, but the rougher nanoparticles enhanced it further.

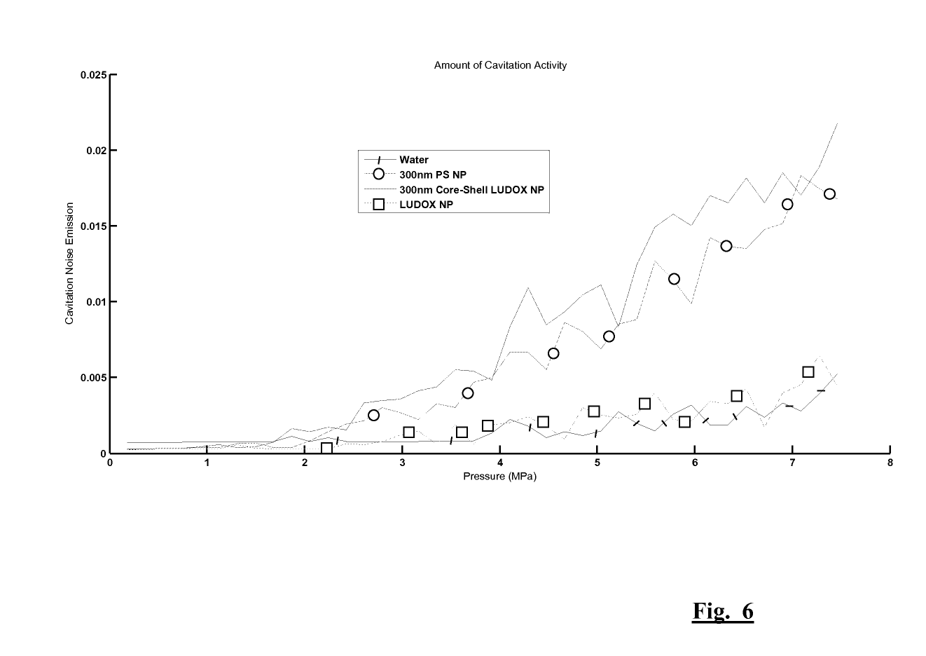

FIG. 6 is a graph showing cavitation noise emissions as a function of peak pressure from the same experiments.

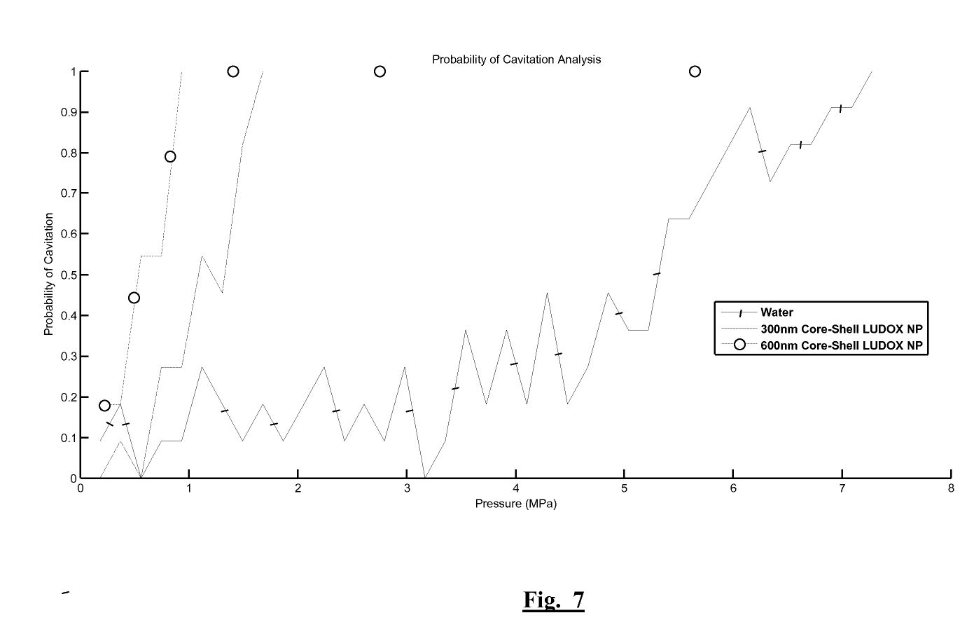

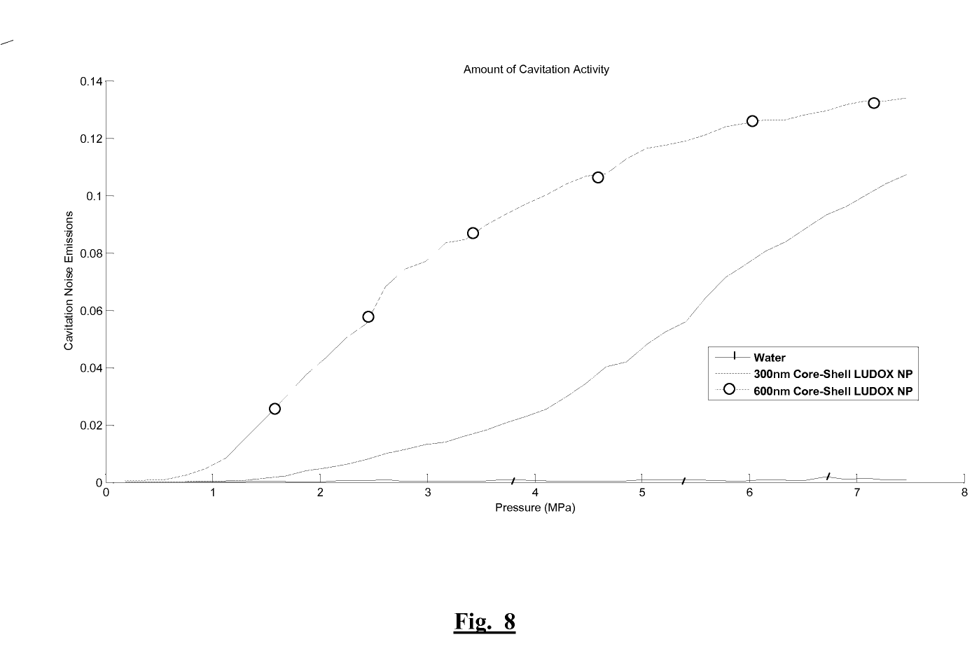

FIG. 7 is a graph similar to FIG. 5 but for nanoparticles of 300 nm and 600 nm diameter, each formed with Ludox™. FIG. 8 shows results from the same experiment expressed as a probability of cavitation similar to FIG. 6. As can be seen, the larger particles increased cavitation more than the smaller ones.

### Emulsification Methods

Nanoparticles according to embodiments of the invention can be produced by either a single or double emulsion method. In one case of the single emulsion method, a suitable water-insoluble polymer (e.g. PLGA, PLA, etc.) is dissolved in an organic solvent which is not miscible with water (e.g. water saturated dichloromethane or chloroform). Pore forming materials (e.g. camphor) and/or active pharmaceutical agents can be added. The resulting solution is then emulsified in a larger volume of an aqueous stabilizer solution to form an oil-in-water (o/w) emulsion. The stabilizers used are usually surface active polymers such as poloxamer or polyvinyl alcohol or o/w-surfactants such as polysorbate 20 or polysorbate 80. Homogenization of the emulsion can be carried out using an ultrasound homogenizer, high pressure homogenizer, high shear homogenizer, or other in order to obtain a nanoemulsion containing nanodroplets of the organic polymer solution dispersed in the aqueous stabilizer solution. The volume ratio between the organic solution to the liquid stabilizer solution is usually between 1:5 to 1:10 (but not limited to). The resulting emulsion is then poured under constant stirring in an excess amount of water or into a low concentrated solution of a water miscible solvent in water (e.g. 2% isopropanol solution) in order to achieve diffusion of the organic solvent from the inner oil phase into the outer water phase and thus harden the particles. The particles are subsequently cleaned and freeze-dried in order to remove any water and to sublime the pore forming material.

In one case of a double emulsion method, a small quantity of water is emulsified in an organic solution of a water-insoluble polymer (e.g. PLA, PLGA) in an organic solvent (not miscible with water) in order to obtain a water-in-oil (w/o)-emulsion. The organic phase may additionally consist of a (w/o)-surfactant (e.g. sorbate 20 or 80) and a pore forming material (e.g. camphor). The volume ratio of aqueous to organic phase is usually between 1:5 and 1:10 (but not limited to). Emulsification is performed using an ultrasound homogenizer, high pressure homogenizer, high shear homogenizer, or other in order to obtain a nanoemulsion containing aqueous nanodroplets dispersed in the organic solution. The resulting w/o-emulsion is then emulsified into an aqueous stabilizer solution to form a (w/o/w) double emulsion. The stabilizers used are usually surface active polymers such as poloxamer or polyvinyl alcohol or o/w-surfactants such as polysorbate 20 or polysorbate 80. Homogenization of the double emulsion can be carried out using an ultrasound homogenizer, high pressure homogenizer, high shear homogenizer, or other in order to obtain a nanoemulsion. The volume ratio between the first emulsion and the liquid stabilizer solution is usually between 1:5 and 1:10 (but not limited to). The resulting double emulsion is then poured under constant stirring in an excess amount of water or into a low concentrated solution of a water miscible solvent in water (e.g. 2% isopropanol solution) in order to achieve diffusion of the organic solvent from the inner oil phase into the outer water phase and thus harden the particles. The particles are the cleaned and freeze-dried in order to remove any water and to sublime the pore forming material.

### Example Single Emulsion:

First, 250 mg camphor was dissolved in 100 ml methylene chloride. Second, 2500 mg PLGA was dissolved in 100 ml camphor solution to yield the final oil phase. 20 ml of the resulting PLGA solution was added to 100 ml of a 50 mg/ml PVA solution at 4° C. and homogenized using ultrasound homogeniser (Sonicator type 7533A) for 4 minutes at 20 W. The resulting emulsion was poured into 500 ml of a 2% isopropanol solution and stirred at 300 rpm for further 4 hours on an ice-bath to evaporate off the methylene chloride and thus harden the particles. The nanoparticles were then collected by centrifugation at 7500 rpm for 20 min at 15° C. After washing three times with deionised water, the particles were re-suspended in deionized water and filled into 20 ml serum tubing glass vials for freeze-drying. Freeze-drying was performed at −15° C. primary drying temperature and +25° C. secondary drying temperature. The vacuum was kept constant at 100 mTorr during drying.

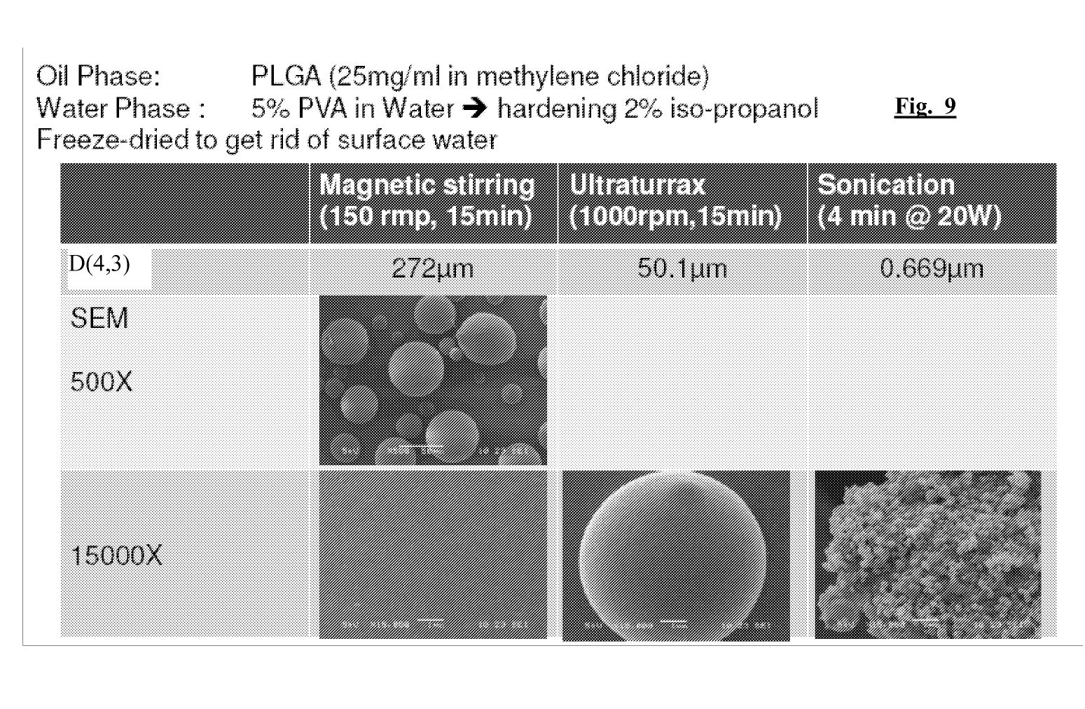

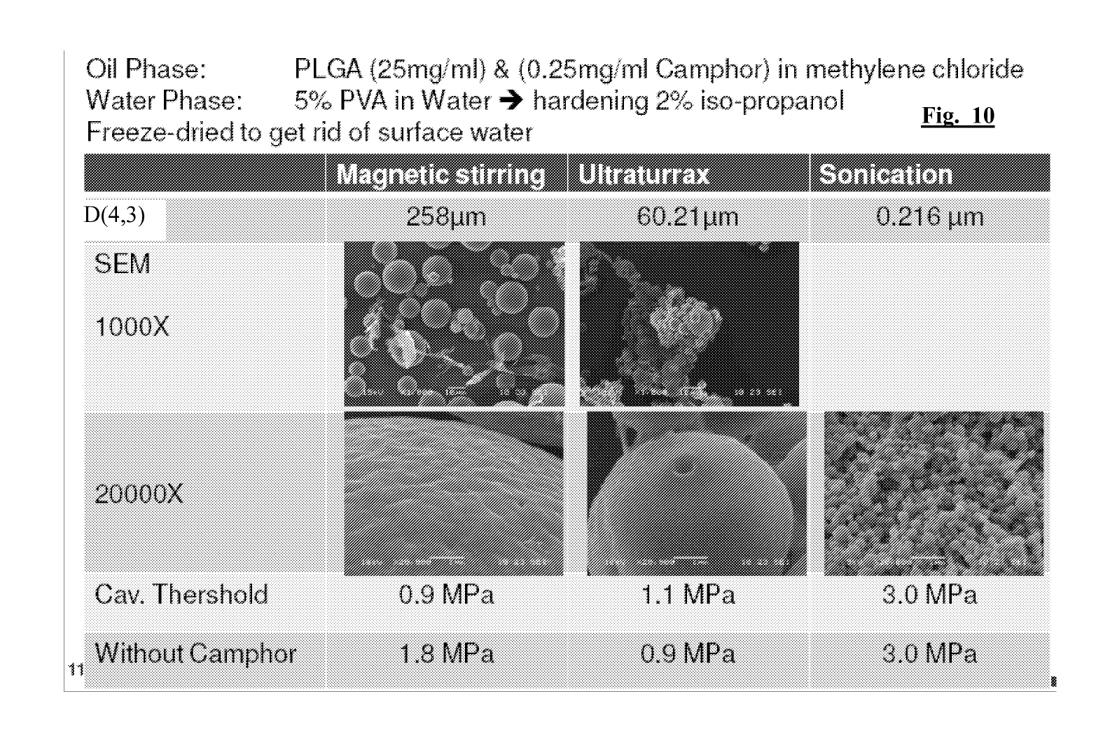

FIGS. 9 and 10 show examples of particles formed by the single emulsion process. It can be seen that the sonication described above results in particles of the right size, whereas methods without sonication result in much larger particles. The surface roughness of the particles produced can be increased by the layer-by-layer deposition of SiO₂ particles as previously described.

### Example Double Emulsion:

First, 250 mg camphor was dissolved in 100 ml methylene chloride. Second, 2500 mg PLGA was dissolved in 100 ml camphor solution to yield the final oil phase. To generate the first w/o emulsion, 1 ml of deionised water was added to 20 ml of the polymer solution and sonicated using an ultrasound homogeniser at 20 W for 30 seconds. The resulting (w/o)-emulsion was then poured into 100 ml of 5% PVA solution (at 4° C.) and homogenized using an ultrasound homogeniser at 20 W to yield a (w/o/w)-double-emulsion. The resulting double emulsion was poured into 500 ml of a 2% isopropanol solution and stirred at 300 rpm for further 4 hours on an ice-bath to evaporate off the methylene chloride and thus harden the particles. The nanoparticles were then collected by centrifugation at 7500 rpm for 20 min at 15° C. After washing three times with deionised water, the particles were re-suspended in deionized water and filled into 20 ml serum tubing glass vials for freeze-drying. Freeze-drying was performed at −15° C. primary drying temperature and +25° C. secondary drying temperature. The vacuum was kept constant at 100 mTorr during drying.

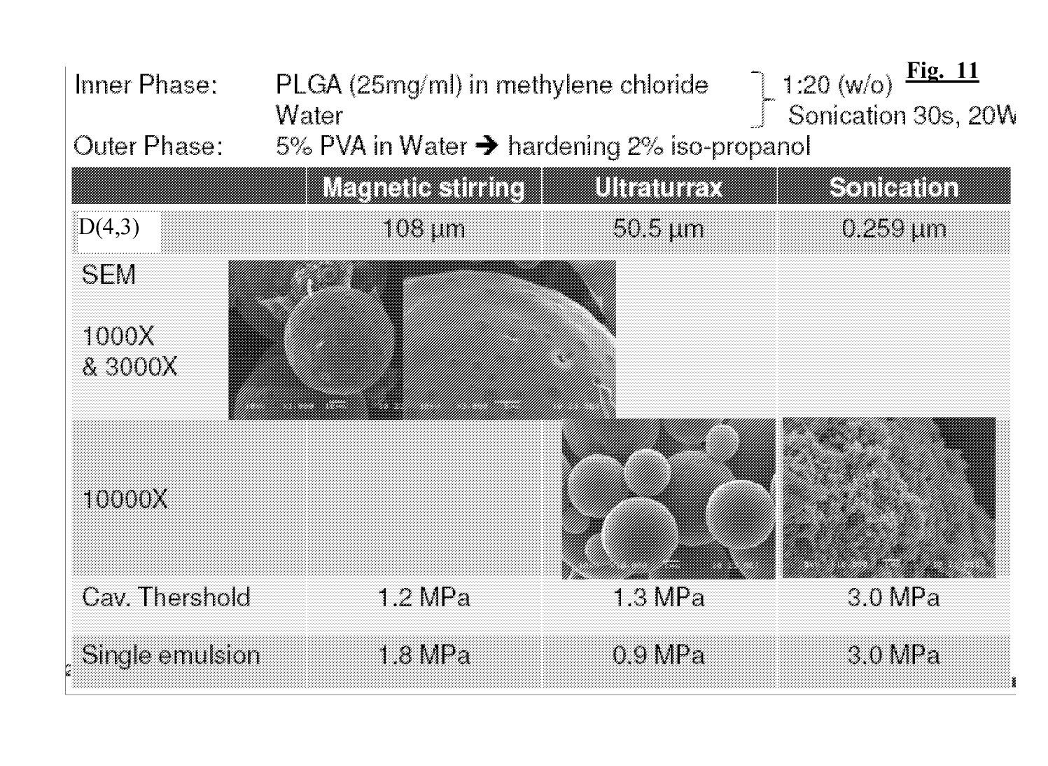

FIG. 11 shows examples of particles formed by the double emulsion process. Again it can be seen that sonication can result in particles of the required size, whereas methods without sonication tend to produce much larger particles. The surface roughness of the particles produced can be increased by the layer-by-layer deposition of SiO₂ particles as previously described.

### Nanoprecipitation (Solvent Displacement):

In this method a water insoluble polymeric material (e.g. PLA, PLGA) is dissolved in an organic solvent which is miscible with water. The resulting solution of polymer in organic solvent can additionally contain, in small quantities, further organic solvents (e.g. benzylbenzoate, benzylalcohol, etc.), liquid oils, or pore forming materials (e.g. camphor), which may or may not be miscible with water. The formulation can further contain a hydrophobic active therapeutic ingredient in solution. Particles are manufactured by adding the organic solution into an excess of aqueous stabilizer solution under constant stirring usually in a volume ratio of 1:10 (but not limited to). The nanoparticles form spontaneously by a nanoprecipitation (solvent displacement) mechanism. Suitable stabilizers are surface active polymers, e.g. polyvinyl alcohols, poloxamers, etc. in concentrations between 0.5% and 5%. In order to achieve particles with a rough surface and air pockets on the surface or inside the nanoparticle, the particles are freeze-dried to remove any water and to sublime the pore forming material.

### Example Nanoprecipitation Method:

125 mg of poly-(D,L-lactide) polymer (PLA) was first dissolved in acetone (25 ml). 0.5 ml of benzyl-benzoate or benzyl alcohol and 12.5 mg of camphor were then added to the acetonic solution. The resulting organic solution was poured in 50 ml of water containing 250 mg of poloxamer under moderate magnetic stirring. The acetone which diffused into the aqueous phase was then removed by stirring at ambient pressure at 4° C. in an ice bath. The steric stabilizer was removed by centrifuging the particles at 15° C., discarding the supernatant and re-suspending the particles in deionized water. This was repeated 3 times. The final particle suspension was subsequently freeze-dried in order to obtain dry nanocapsules and to sublime the camphor present in the particle formulation. Sublimation of the camphor resulted in pore formation and increase in surface roughness after freeze-drying.

### Spray-Freeze-Drying

In this method, nanoparticles can be manufactured by any form of spray-freeze-drying from an initial liquid feed comprising between as low as about 0.01% (and lower) and about 10% concentration of the particle constituent or constituents in solution, emulsion or in suspension. In particular, the feed liquid may be an aqueous solution or suspension or an organic solvent having, in solution or in suspension, the particle constituents, including the pharmacologically active ingredient and any necessary excipients or stabilisers. The feed liquid may be of emulsion type such as a single-emulsion, double-emulsion or micro-emulsion. One or more suitable solvents or dispersion medium may be used for the preparation of the emulsion. The solvents or dispersion mediums may contain suitable dissolved substances to adjust the properties of the feed liquid, such as pH, tonicity, viscosity, surface tension etc. The spray-freeze-drying from an initial feed liquid may be combined with subsequent processing steps such as ultrasound homogenization (sonication), compressing, milling, sieving, spray-coating, or nanoencapsulation. Particles may also be produced by any combination of the above techniques.

### Example 1 for Spray-Freeze-Drying:

Dissolution of a biodegradable polymer (e.g. PLGA) in an organic solvent (e.g. acetonitrile) in low concentration (e.g. <1%) with subsequent atomization of the liquid solution into a container with a suitable cryogenic liquid (e.g. liquid nitrogen) using a suitable nozzle system (e.g. ultrasound atomizer, two-fluid nozzle, monodisperse droplet generator, etc.). The droplets immediately freeze upon impact with the cryogenic liquid and are subsequently transferred onto the pre-cooled shelves of a freeze-drying system. Freeze-drying is performed at low temperature and pressure, meaning below the melting temperature of the solvent and the collapse temperature of the formulation. After freeze-drying, the dry nanoparticles are used either directly without further processing or particle size is further reduced by suspending the biodegradable powder after SFD in a suitable dispersion medium and subjecting the suspension to ultrasound homogenization for some minutes. The latter procedure is follow by a suitable drying step such as an additional freeze-drying cycle to remove the dispersion medium and to obtain a dry product.

### Example 2 for Spray-Freeze-Drying:

20 mg PLA was dissolved in 20 g p-Xylene under constant stirring using a magnetic stirrer. Spray-freezing was performed by atomization of the liquid solution at a flow rate of 1 ml/min into a stainless steel bowl filled with liquid nitrogen using 60 kHz ultrasound nozzle (Sono Tek, USA). The power of the nozzle was set to 4 Watts. At the end of the spray-freezing procedure, the stainless steel bowls with the spray-frozen droplets were topped up with liquid nitrogen and transferred onto the pre-cooled shelves (−40° C.) of laboratory freeze-drying system (FTS Lyostar 1, USA). Freeze-drying was performed at a primary drying temperature of −15° C. and a secondary drying temperature of +25° C. At the end of the freeze-drying cycles the final dry powder was transferred from the stainless steel bowls into 20 ml serum tubing glass vial in the humidity controlled environment (0.5% relative humidity at 20° C.) of a glove box.

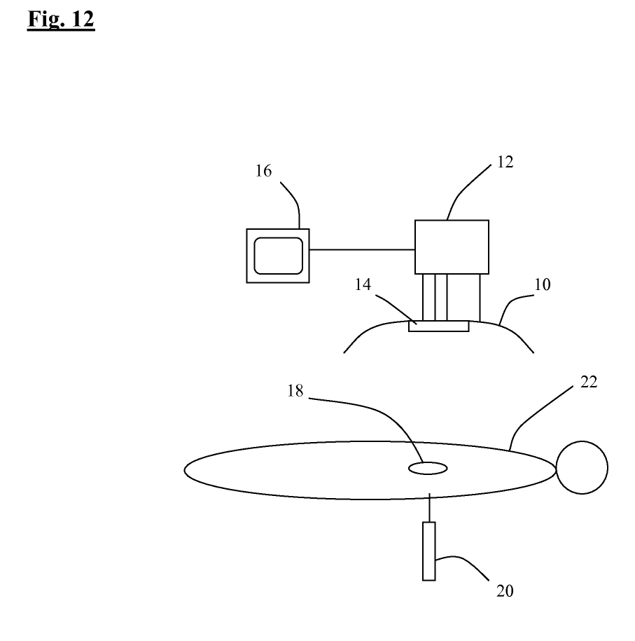

Referring to FIG. 12, particles made according to any of the methods described above and having the desired size and surface roughness can be used to induce cavitation in tissue under ultrasound insonation. The cavitation can be used for imaging purposes or to enhance the delivery of therapeutic substances to the tissue. FIG. 8 shows an ultrasound system that performs both of these functions. The ultrasound system comprises a source 10 of ultrasound in the form of an ultrasound transducer, which is controlled by a controller 12 in the form of a computer. An ultrasound detector array 14 is located at the centre of the ultrasound transducer 10 and is arranged to detect passively ultrasound at a higher frequency as emitted from cavitation in tissue. A display screen 16 is connected to the computer 12. The computer 12 comprises memory and a processor and is arranged to control the operation of the ultrasound transducer 10, to process the signals from the detector array 14 and to generate images for display on the screen 16. The transducer 10 is arranged to insonate an insonation region 18, and the detector array 14 is arranged to detect ultrasound coming from the insonation region 18. A source 20 of nanoparticles is provided to enable the infusion of nanoparticles into a patient 22, in this example into the patient's liver, at a position located in the insonation region 18. The computer is arranged to control the transducer so as to generate ultrasound at any frequency within the range 100 kHz-10 MHz.

In one mode of operation nanoparticles are infused into the patient's liver. Due to the size of the particles they enter and accumulate in the vasculature of any cancer tumour in the liver. The computer 12 is then arranged to control the transducer 10 to insonate the insonation region 18, and to process the signals from the detector array 14. The surface roughness of the nanoparticles induces cavitation which is therefore concentrated in the areas where the particles have accumulated, and the cavitation is detected and imaged by the computer 12. The nanoparticles can be made by any of the processes described above.

In another mode of operation the nanoparticles are mixed with a therapeutic substance which is formed as, or carried on, nanoparticles of a similar size, i.e. in the range 50-800 nm for optimum take-up, and preferably 100-600 nm. The therapeutic substance may be any appropriate type of drug, for example an anti-cancer agent, siRNA, adenoviral vectors, or any small molecule drugs. The nanoparticles carrying the therapeutic substance in this case do not need to induce cavitation and so their surface roughness is not critical. The mixture is infused into the patient and the sonosensitive nanoparticles and the therapeutic substance carrying nanoparticles, because they are of similar size, will tend to accumulate in the same parts of the patient as each other. Ultrasound insonation by the transducer 10 therefore causes cavitation which can be imaged using the detector array 14, and this will give an indication of the location of the sonosensitive nanoparticles from the mixture, and hence also of the therapeutic substance carrying nanoparticles. As the imaging can be performed in real time, this allows the real time monitoring of accumulation of the therapeutic substance carrying nanoparticles in the cancerous tissue to be treated, and hence real time imaging of the delivery of the therapeutic substance to the area to which it is targeted. In this mode of operation, it will be appreciated that the cavitation induced by the sonosensitive nanoparticles will also enhance the delivery of the therapeutic substance to the target area.

In a further mode of operation, a therapeutic substance is encapsulated within, or otherwise carried on, the sonosensitive nanoparticles, which are delivered to the target site. In this case the drug carrying nanoparticles themselves act to induce cavitation when insonated with ultrasound because of their surface roughness. This again allows the delivery of the drug to be monitored using imaging of the cavitation induced by the nanoparticles. Simultaneously the cavitation will also enhance the delivery of the therapeutic substance at the target site.

In a further embodiment the method includes both mapping the location of the nanoparticles and enhancing and/or mapping of delivery of the therapeutic substance to tissue. In this case nanoparticles carrying the drug and each having surface features of two different depths are used. The nanoparticles are infused into the patient and during the infusion the nanoparticles are insonated with ultrasound matched to a first one of the features sizes (depth or other scale) to cause cavitation. This cavitation is imaged as described above so that the infusion process can be monitored. Then when the nanoparticles have accumulated in the desired location, they are insonated with ultrasound of a second, different frequency matched to the surface features of the second depth or scale. This causes cavitation bubbles having different characteristics from those of the first insonation, for example of a different size, which is arranged to rupture the nanoparticles.

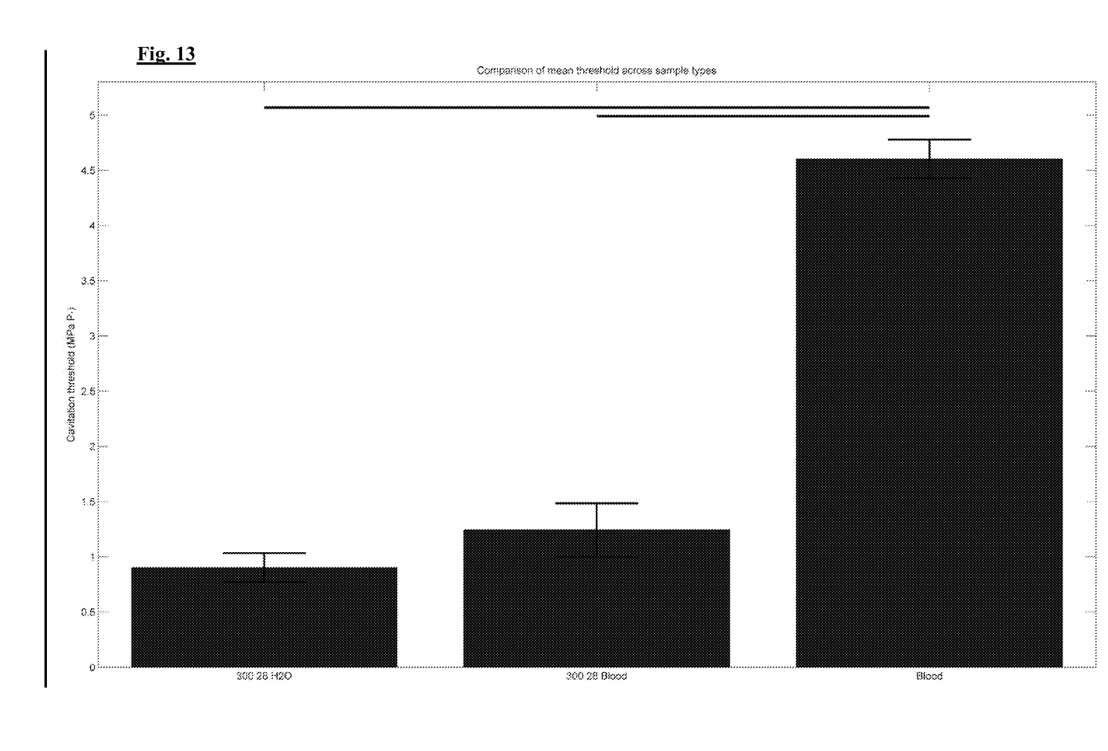

Referring to FIG. 13, nano-particles were made using the layer-by-layer method described above with a particle size of 300 nm and with coating particles of 28 nm. Samples of water containing these particles, blood containing these particles, and blood with no particles were insonated with ultrasound at 1 MHz. The results show that the cavitation threshold is significantly reduced for the particles in blood, as well as for the particles in water, compared with the blood without particles. This suggests that the hydrophobicity and surface roughness of the particles is not affected when the nanoparticles become coated with plasma proteins.

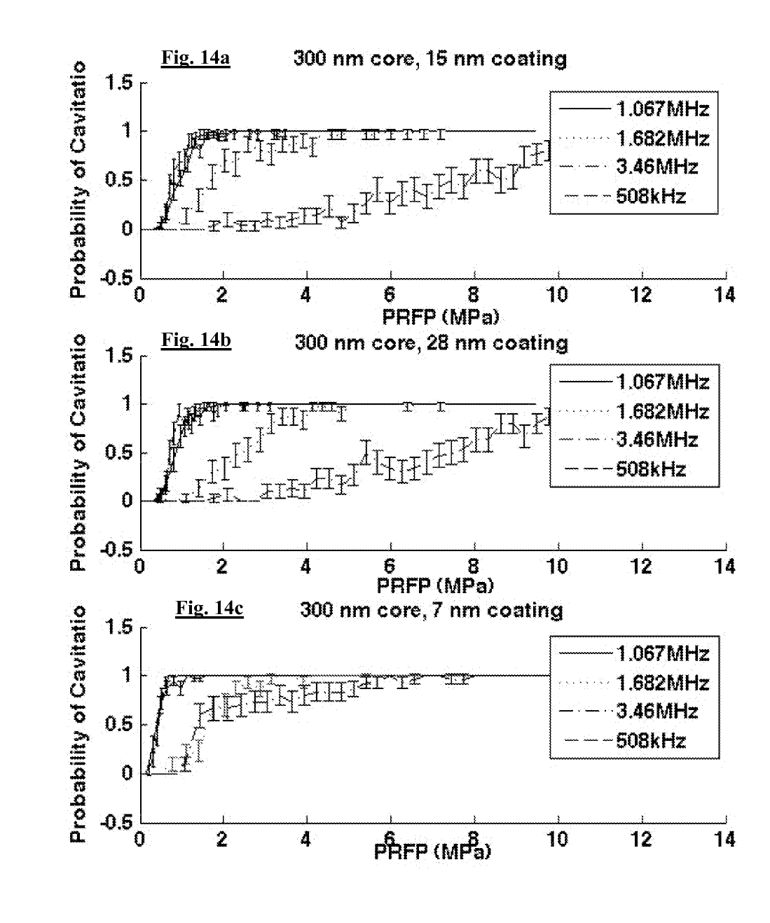

Referring to FIG. 14a, similar particles with 300 nm core size and 15 nm coating particles were placed in water, and the water insonated with ultrasound at 508 kHz, 1.067 MHz, 1.682 MHz and 3.46 MHz. It can be seen that, with increasing peak pressure, cavitation was induced at lowest peak pressure at 508 kHz and slightly higher peak pressure at 1.067 MHz, at higher pressure at 1.682 MHz, and at still higher pressure at 3.46 MHz.

Referring to FIG. 14b, the same measurements were made with particles which were the same except for the coating particle size which was 28 nm. As can be seen, the reduction in threshold pressure is less marked, in particular at 1.682 MHz.

Referring to FIG. 14c, the same measurements were made with particles which were the same except for the coating particle size which was 7 nm. As can be seen, the reduction in threshold pressure is much more marked for all frequencies.

Comparing the results for 3.46 MHz between the 300 nm core 7 nm coating and 300 nm core 28 nm coating, the probability of cavitation is considerably greater for a lower pressure for the 7 nm coating. This suggests that for the same curvature of template (i.e. core particle size), use of smaller coating particles results in the entrapment of smaller nuclei which are better suited for the initiation of inertial cavitation at higher frequencies.

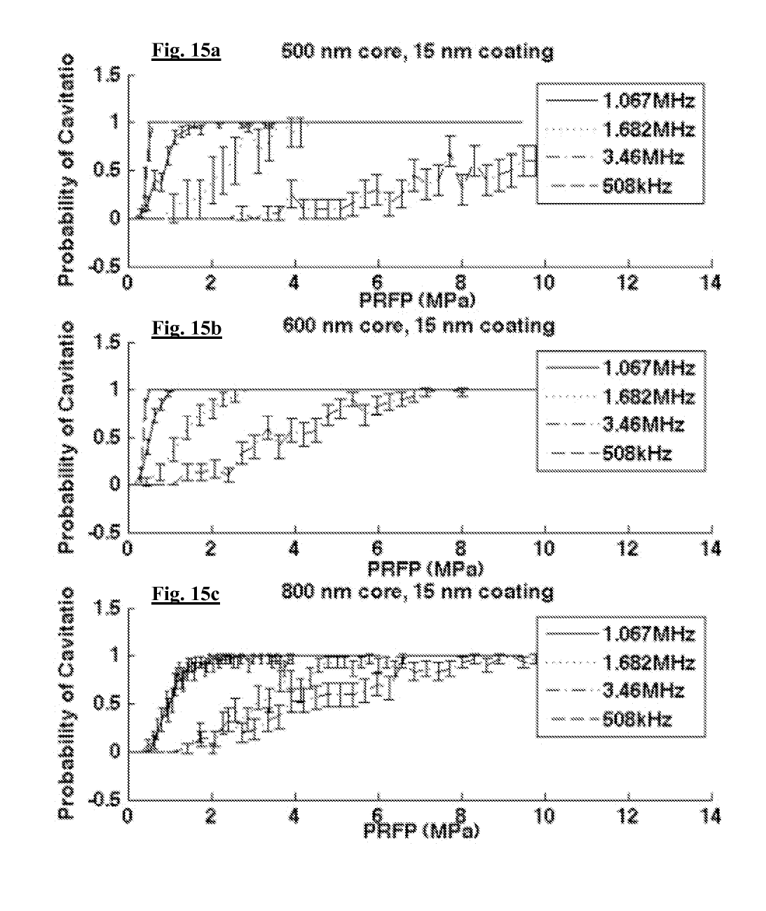

Referring to FIG. 15a, similar particles, but this time with 500 nm core size and 15 nm coating particles were placed in water, and the water insonated with ultrasound at 508 kHz, 1.067 MHz, 1.682 MHz and 3.46 MHz. It can be seen that, as with the results of FIG. 14a, the reduction in threshold pressure is greater with decreasing frequency.

Referring to FIG. 15b, the core size was changed to 600 nm with other parameters remaining the same. It can be seen that, at the higher frequencies, the threshold pressure is reduced further. Referring to FIG. 15c, for core size of 800 nm a further reduction in threshold pressure at 3.46 MHz can be seen.

Looking at the results for 3.46 MHz for all core sizes with the 15 nm coating, the probability of cavitation becomes increasingly higher at lower pressures (i.e. more inertial cavitation can be generated more easily) as the template (core) size is increased from 300 nm, to 500 nm, to 600 nm to 800 nm. This strongly suggests that the decreasing curvature resulting from the larger templates results in the entrapment of smaller nuclei which are better suited for the initiation of inertial cavitation at higher frequencies.

1. A nanoparticle for inducing cavitation in a medium under insonation, the nanoparticle having a diameter in the range from 10 to 1000 nm and surface features having a depth in the range from 5 to 50 nm.

2. A nanoparticle for the treatment of cancer in a body, the nanoparticle having a diameter in the range from 10 to 1000 nm and surface features having a depth in the range from 5 to 50 nm, whereby the nanoparticle is arranged to enhance cavitation in the body when the body is insonated with pressure waves.

3. A system for treating cancerous tissue, the system comprising a source of pressure waves and nanoparticles for delivery to the tissue, the nanoparticles having a diameter in the range from 10 to 1000 nm and surface features having a depth in the range from 5 to 50 nm, whereby the nanoparticles are arranged to enhance cavitation in the tissue when the tissue is insonated with pressure waves from the source.

4. A method of controlling cavitation in tissue, the method comprising delivering nanoparticles to the tissue, the nanoparticles having a diameter in the range from 10 to 1000 nm and surface features having a depth in the range from 5 to 50 nm, and insonating the tissue with pressure waves.

5. A method of imaging an object, the method comprising delivering nanoparticles to the object, the nanoparticles having a diameter in the range from 10 to 1000 nm and surface features having a depth in the range from 5 to 50 nm, and insonating the object with pressure waves such that the nanoparticles induce cavitation in the object, detecting pressure waves generated by the cavitation by means of a detector, and processing signals from the detector to generate an image of the object.

6. A method of monitoring the delivery of a therapeutic substance to tissue, the method comprising delivering the therapeutic substance and nanoparticles to the tissue, the nanoparticles having a diameter in the range from 10 to 1000 nm and surface features having a depth in the range from 5 to 50 nm, and insonating the tissue with pressure waves such that the nanoparticles induce cavitation in the tissue, detecting pressure waves generated by the cavitation by means of a detector, and processing signals from the detector to monitor the delivery.

7. A method of delivering a therapeutic substance to tissue, the method comprising delivering the therapeutic substance and nanoparticles to the tissue, the nanoparticles having a diameter in the range from 10 to 1000 nm and surface features having a depth in the range from 5 to 50 nm, and insonating the tissue with pressure waves.

8. A nanoparticle, system or method according to claim 1 wherein the surface features are formed by spheres or part-spheres with diameter of 5-50 nm, or depressions with depth of 5-50 nm, and or width of 5-50 nm.

9. A nanoparticle, system or method according to claim 1 wherein the nanoparticles are hydrophobic.

10. A nanoparticle, system or method according to claim 1 wherein the nanoparticles carry a drug.

11. A nanoparticle, system or method according to claim 1 wherein the nanoparticles are hollow forming nanocapsules.

12. A nanoparticle, system or method according to claim 1 wherein the nanoparticles are freeze-dried or spray-freeze-dried or spray-dried.

13. A nanoparticle, system or method according to claim 1 wherein the nanoparticles are each formed by providing a core and forming a shell on the core.

14. A nanoparticle, system or method according to claim 13 wherein the core is of polystyrene.

15. A nanoparticle, system or method according to claim 13 wherein the shell is formed at least partly from silicon dioxide or titanium dioxide.

16. A nanoparticle, system or method according to claim 13 wherein the core is removed to leave a hollow shell.

17. A nanoparticle, system or method according to claim 13 wherein the shell is formed at least partly from particles having a diameter in the range 5 to 50 nm so as to provide the surface features.

18. A method according to claim 4 wherein the pressure waves have a frequency of at least 100 kHz.

19. A method according to claim 4 wherein the pressure waves have a frequency of at least 500 kHz.

20. A method according to claim 4 wherein the pressure waves have a frequency of not more than 10 MHz.

21. A method according to claim 4 wherein the pressure waves have a frequency of not more than 5 MHz.

22. A system according to claim 3 wherein the pressure waves have a frequency of at least 100 kHz.

23. A system according to claim 3 wherein the pressure waves have a frequency of at least 500 kHz.

24. A system according to claim 3 wherein the pressure waves have a frequency of not more than 10 MHz.

25. A system according to claim 3 wherein the pressure waves have a frequency of not more than 5 MHz.
